(19) United States
(12) Patent Application Publication
(10) Pub. No.: US 2026/0229365 A1
(43) Pub. Date: Aug. 6, 2026

(54) TRAINING DEVICE, TRAINING METHOD, AND NON-TRANSITORY COMPUTER-READABLE MEDIUM

(71) Applicant: NEC Corporation, Tokyo (JP)

(72) Inventors: Chenhui HUANG, Tokyo (JP); Fumiyuki NIHEY, Tokyo (JP); Masataka ANDOU, Tokyo (JP)

(73) Assignee: NEC Corporation, Tokyo (JP)

(21) Appl. No.: 19/447,064

(22) Filed: Jan. 13, 2026

(30) Foreign Application Priority Data
Jan. 31, 2025 (JP) 2025-014523

(51) Int. Cl. G16H 50/30

(52) U.S. Cl. CPC G16H 50/30

(57) ABSTRACT

A training device according to the present disclosure includes a generation unit that generates analysis data indicating a variation of measurement data in a life cycle, based on a timing at which the measurement data regarding a first subject whose disease risk is known in advance is generated and a training unit that machine learns a training model for estimating the disease risk using the analysis data as an input, using the disease risk of the first subject known in advance and the analysis data regarding the first subject as training data. This makes it possible to support decision making of the first subject regarding healthcare.

### INCORPORATION BY REFERENCE

This application is based upon and claims the benefit of priority from Japanese patent application No. 2025-014523, filed on January 31, 2025, the disclosure of which is incorporated herein in its entirety by reference.

### TECHNICAL FIELD

The present disclosure relates to a training device, a training method, and a program.

### BACKGROUND ART

With the spread of smartphone terminals and wearable terminals, it is possible to collect various life logs related to daily life of users of the wearable terminals. Here, the life log is data such as a heart rate, a pulse, or a body temperature of the user of the wearable terminal or the like. In the future, it is desired to grasp a health condition of the user using the life log and further estimate a disease risk.

JP 2017-006745 A discloses a configuration of a health information processing device that analyzes genome information, biological information, and behavior information of a user to estimate a future health risk of the user.

### SUMMARY

With an increase in interest in healthcare, it is desired to estimate a disease risk with high accuracy using a large amount of collected sensor data or life logs in the future. It is estimated that the disease risk changes depending on a lifestyle of an individual. Therefore, it is desired to estimate the disease risk that changes depending on the lifestyle of the individual with high accuracy.

An example object of the present disclosure is to provide a training device, a training method, and a program capable of assuming a disease risk in consideration of an individual lifestyle.

A training device according to an example aspect of the present disclosure includes a generation unit that generates analysis data indicating a variation of measurement data in a life cycle, based on a timing at which the measurement data regarding a first subject whose disease risk is known in advance is generated and a training unit that machine learns a training model for estimating the disease risk using the analysis data as an input, using the disease risk of the first subject known in advance and the analysis data regarding the first subject as training data.

A training method according to an example aspect of the present disclosure includes generating analysis data indicating a variation of measurement data in a life cycle, based on a timing at which the measurement data regarding a first subject whose disease risk is known in advance is generated and estimating the disease risk using the analysis data as an input, using the disease risk of the first subject known in advance and the analysis data regarding the first subject as training data.

A program according to an example aspect of the present disclosure causes a computer to execute generating analysis data indicating a variation of measurement data in a life cycle, based on a timing at which the measurement data regarding a first subject whose disease risk is known in advance is generated and estimating the disease risk using the analysis data as an input, using the disease risk of the first subject known in advance and the analysis data regarding the first subject as training data.

According to the present disclosure, a training device, a training method, and a program capable of assuming a disease risk in consideration of an individual lifestyle can be provided.

### BRIEF DESCRIPTION OF THE DRAWINGS

FIG. 1 illustrates a configuration example of a training device;

FIG. 2 illustrates a flow of processing related to a training method executed by the training device;

FIG. 3 illustrates a configuration example of the training device;

FIG. 4 illustrates a flow of training processing executed by the training device;

FIG. 5 illustrates an image regarding measurement data;

FIG. 6 illustrates an image of the measurement data classified for each day of the week;

FIG. 7 illustrates an image regarding statistical data;

FIG. 8 illustrates an example of analysis data;

FIG. 9 illustrates an example of the analysis data;

FIG. 10 illustrates a flow of disease risk estimation processing; and

FIG. 11 is a block diagram illustrating a training device configuration example.

### EXAMPLE EMBODIMENT

### First Example Embodiment

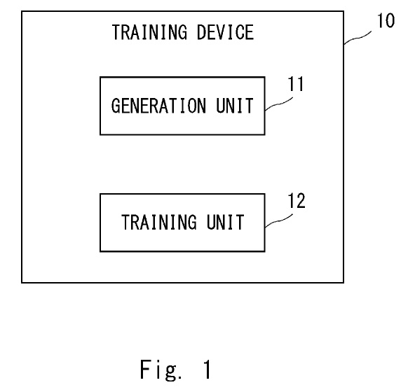

FIG. 1 illustrates a configuration example of a training device 10. The training device 10 may be a computer device that operates by a processor executing a program stored in a memory. For example, the training device 10 may be a server device or a personal computer device.

The training device 10 includes a generation unit 11 and a training unit 12. The generation unit 11 and the training unit 12 may be software components or modules whose processing is carried out by causing the processor to execute the program stored in the memory. Alternatively, the generation unit 11 and the training unit 12 may be hardware components such as circuits or chips.

As illustrated in FIG. 1, the generation unit 11 and the training unit 12 may be included in the single training device 10 or may be respectively included in different computer devices. In a case where the generation unit 11 and the training unit 12 are respectively included in the different computer devices, each computer device may perform communication via a network. The network may be a so-called intranet that is a closed network or may be the so-called Internet that is an open network. In a case where the generation unit 11 and the training unit 12 are included in the different computer devices, the generation unit 11 and the training unit 12 may be regarded as elements constituting a training system.

The generation unit 11 generates analysis data indicating a variation of measurement data in a life cycle, based on a timing at which the measurement data regarding a first subject whose disease risk is known in advance is generated.

The disease risk may be a possibility of having some disease in the future. The disease risk may be referred to as a health risk. The disease risk may be, for example, a risk of diabetes, hypertension, or the like or may be a risk of frailty. A case where the disease risk is known in advance may be a case where it is determined that there is the disease risk, in an answer result of a questionnaire for examining the disease risk or the like. Alternatively, a case where the disease risk is known in advance may be a case where it is determined that there is the disease risk in a medical examination or the like.

The measurement data may be, for example, data that can be measured by a smartphone terminal held by the first subject, a wearable sensor worn by the first subject, or the like. Alternatively, the measurement data may be data measured by a measurement device such as a weight scale or a blood pressure monitor. Items of the measurement data may be, for example, the number of steps, a moving speed, a walking time, a stride length, a pitch, a walking distance, Metabolic Equivalent of Tasks (METs), total calorie consumption, a lying time, a sitting time, a weight, a blood pressure, or the like. The generation unit 11 collects the measurement data from the plurality of first subjects. Lifestyles of the plurality of first subjects vary. In addition, ages of the plurality of first subjects may vary.

In addition, it is assumed that the measurement data be associated with a timing at which the measurement data is measured or generated. Alternatively, the measurement data may include information indicating the timing at which the measurement data is measured or generated. The timing may be, for example, information indicating a date and time.

The measurement data may be also referred to as life log data, sensor data, health data, or the like. The generation unit 11 may receive the measurement data from the smartphone terminal, the wearable sensor, the measurement device, or the like via the network. Alternatively, the generation unit 11 may receive the measurement data from the wearable sensor, the measurement device, or the like via the smartphone terminal and the network. In this case, the smartphone terminal may perform near field communication, wireless Local Area Network (LAN) communication, or the like with the wearable sensor and the measurement device.

The life cycle may be, for example, one day, one week, one month, or one year. For example, in a case where the life cycle is one day, the variation of the measurement data in the life cycle may be a variation in one day of measurement data classified for each predetermined period, such as from 6:00 to 12:00, from 12:00 to 18:00, and from 18:00 to 6:00 on the next day. In a case where the life cycle is one week, the variation of the measurement data may be a variation in one week of measurement data classified for each day of the week. In a case where the life cycle is one month, the variation of the measurement data may be a variation in one month of measurement data classified for each day. In a case where the life cycle is one month, the variation of the measurement data may be obtained by classifying the measurement data for each predetermined period, for example, from a first day to a seventh day, from an eighth day to a fourteenth day, from a fifteenth day to a twenty first day, and from a twenty second day to a thirty first day. In a case where the life cycle is one year, the variation of the measurement data may be a variation in one year of measurement data classified for each month. Alternatively, in a case where the life cycle is one year, the variation of the measurement data may be a variation in one year of measurement data classified for each predetermined period, for example, from January to March, from April to June, from July to September, and from October to December. Alternatively, in a case where the life cycle is one year, the variation of the measurement data may be a variation in one year of measurement data classified into spring, summer, autumn, and winter.

The training unit 12 uses the disease risk of the first subject known in advance and the analysis data regarding the first subject as training data and machine learns a training model that estimates the disease risk using the analysis data as an input. That is, the training unit 12 generates the training model that estimates the disease risk using the analysis data as the input, by performing machine learning.

For example, from the variation of the measurement data such as the number of steps or the walking time, a decrease in an exercise volume may be confirmed. It is generally known that the decrease in the exercise volume may be the disease risk of diabetes, a high blood pressure, or the like, and it can be said that there is a correlation between the analysis data and the disease risk. It is generally known that a weight loss, a walking speed slower than a predetermined speed, or the like may constitute frailty, from the variation of the measurement data of the weight, the walking speed (moving speed), or the like, and it can be said that there is the correlation between the analysis data and the disease risk. The training model for estimating the disease risk using the analysis data as an input is generated by performing learning using a machine learning algorithm or a machine learning model using the analysis data and the disease risk having the correlation as training data.

Machine learning is a technology for achieving Artificial Intelligence (AI). The machine learning may be divided into, for example, supervised learning, unsupervised learning, and reinforcement learning. Supervised learning and unsupervised learning may be used in combination. Here, the training data may be also referred to as teacher data, training data, or the like.

A neural network may be used as the machine learning algorithm or the machine learning model. The neural network includes a plurality of artificial neurons and has synapses connecting the neurons. Each synapse has a weight. In a case where an input is received, such a neural network performs calculation using a weight associated with each synapse, and performs output according to the input. A model representing the connection relationship between the neuron and the synapse is stored in the memory in a form of software, for example. Alternatively, the model may be achieved as a dedicated circuit. Similarly, the weight of each synapse is also stored in the memory in the form of software. A circuit representing a weight may be mounted in a dedicated circuit. In a case where the AI is configured using a plurality of models, not all the models are necessarily stored in the same memory. There are various models using such a neural network, and the AI may be achieved by adopting and replacing various models such as a Transformer, a Convolutional Neural Network (CNN), or a Recurrent Neural Network (RNN).

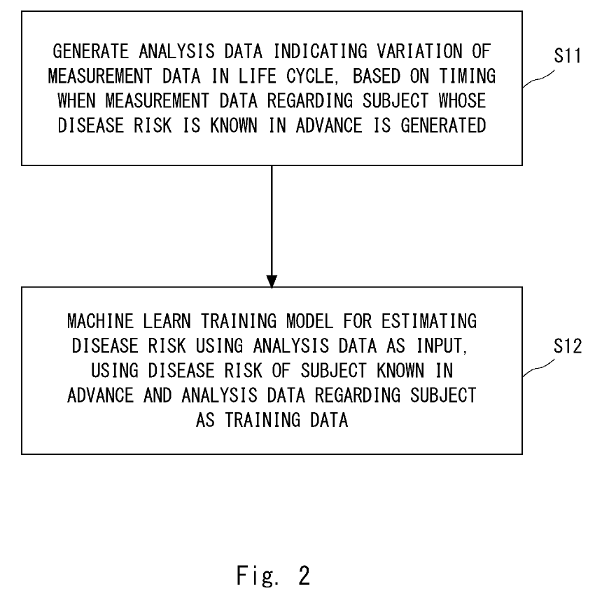

FIG. 2 illustrates a flow of processing related to a training method executed by the training device 10. First, the generation unit 11 generates the analysis data indicating the variation of the measurement data in the life cycle, based on the timing at which the measurement data regarding the first subject whose disease risk is known in advance is generated (S11). Next, the training unit 12 uses the disease risk of the first subject known in advance and the analysis data regarding the first subject as the training data and machine learns the training model that estimates the disease risk using the analysis data as an input (S12).

As described above, the training device 10 can generate the training model that estimates the disease risk by inputting the analysis data, by performing machine learning using the analysis data indicating the variation of the measurement data in the life cycle and the disease risk as the training data. In machine learning, measurement data of various individuals having different lifestyles is used. As a result, the training device 10 can generate the training model that can estimate the disease risk in consideration of an individual lifestyle. By estimating the disease risk, it is possible to support the first subject in decision making regarding healthcare, for example, a change in the lifestyle or the like.

### Second Example Embodiment

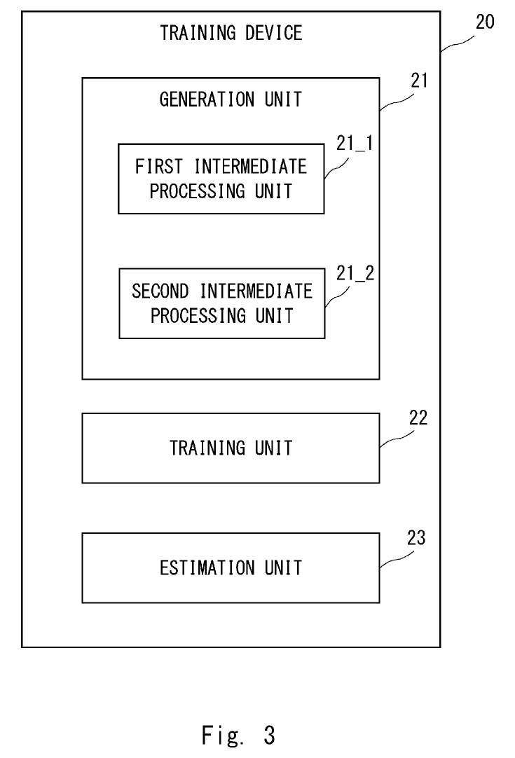

FIG. 3 illustrates a configuration example of a training device 20. The training device 20 has a configuration in which an estimation unit 23 is added to the training device 10 in FIG. 1. Since a generation unit 21 and a training unit 22 included in the training device 20 respectively correspond to the generation unit 11 and the training unit 12 of the training device 10, detailed description of processing, functions, or the like similar to those of the generation unit 11 and the training unit 12 is omitted.

The generation unit 21, the training unit 22, and the estimation unit 23 may be software components or modules whose processing is carried out by causing a processor to execute a program stored in a memory. Alternatively, the generation unit 21, the training unit 22, and the estimation unit 23 may be hardware components such as circuits or chips.

The generation unit 21 includes a first intermediate processing unit 21_1 and a second intermediate processing unit 21_2. Functions and operations of the first intermediate processing unit 21_1 and the second intermediate processing unit 21_2 will be described later in detail.

The estimation unit 23 estimates a disease risk, by inputting analysis data indicating a variation of measurement data into a training model machine learned by the training unit 22. Here, the training unit 22 related to a training phase and the estimation unit 23 related to an inference phase may be included in the same device as illustrated in FIG. 3 or may be included in different devices. That is, training using training data and inference using a trained model may be performed by different devices.

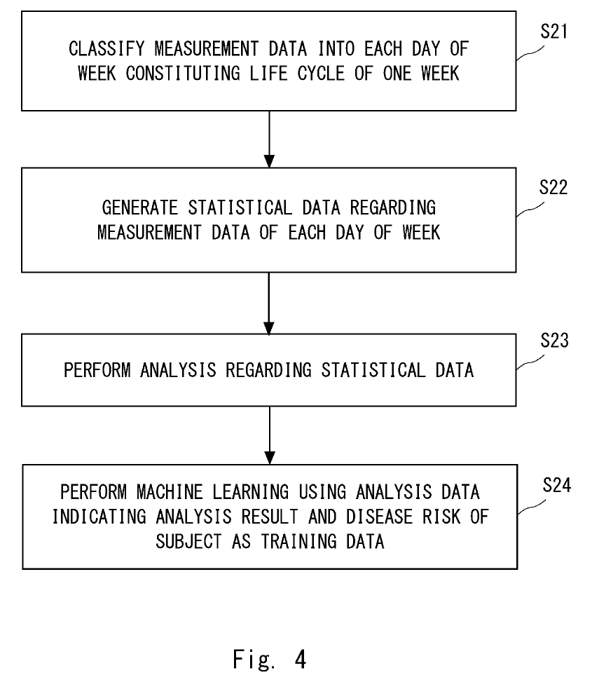

FIG. 4 illustrates a flow of training processing executed by the training device 20. First, the first intermediate processing unit 21_1 classifies the measurement data into each day of the week constituting a life cycle in one week (S21).

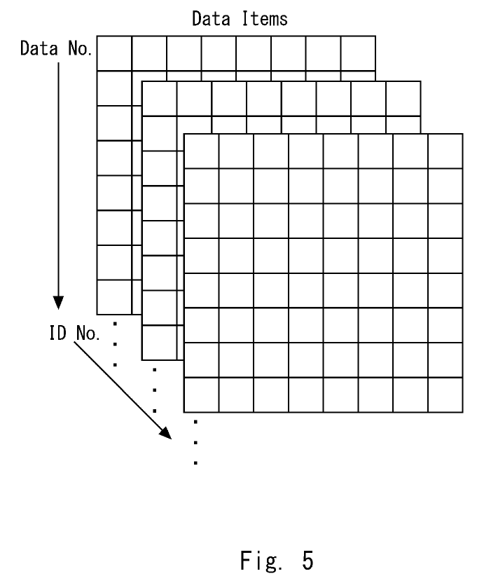

FIG. 5 illustrates an image regarding the measurement data. In FIG. 5, three planes including a plurality of quadrangles are illustrated. Each plane is identified by an Identification (ID). The ID is used to identify a set of the measurement data. Specifically, it is assumed that pieces of data regarding different subjects be associated with the respective planes. The ID may indicate identification information of the subject. The plurality of quadrangles included in the same plane indicates a plurality of pieces of measurement data regarding the same subject. The subject may be a person who has provided the training data.

It is assumed that the plurality of quadrangles included in each plane be associated with a Data Item (data item) and information indicating a timing at which the measurement data is generated (Data No). For example, it is assumed that, in each plane, pieces of measurement data of the same data item generated at different timings be indicated in one column.

For example, each column on one plane may indicate the data item such as the number of steps, a moving speed, a walking time, a stride length, a pitch, a walking distance, METs, total calorie consumption, a lying time, a sitting time, a weight, or time-series data of a blood pressure or the like. The timing associated with each item may be associated with a date and time including a day of the week.

Although data for three subjects is illustrated in FIG. 5, the number of subjects is not limited to three and may be more than three. The number of pieces of data measured for each subject may be the same as illustrated in FIG. 5 or may be different for each subject. Moreover, the data item may be different for each subject. Moreover, a timing at which the data is generated may be different for each data item. Moreover, the number of pieces of measured data may be different for each data item.

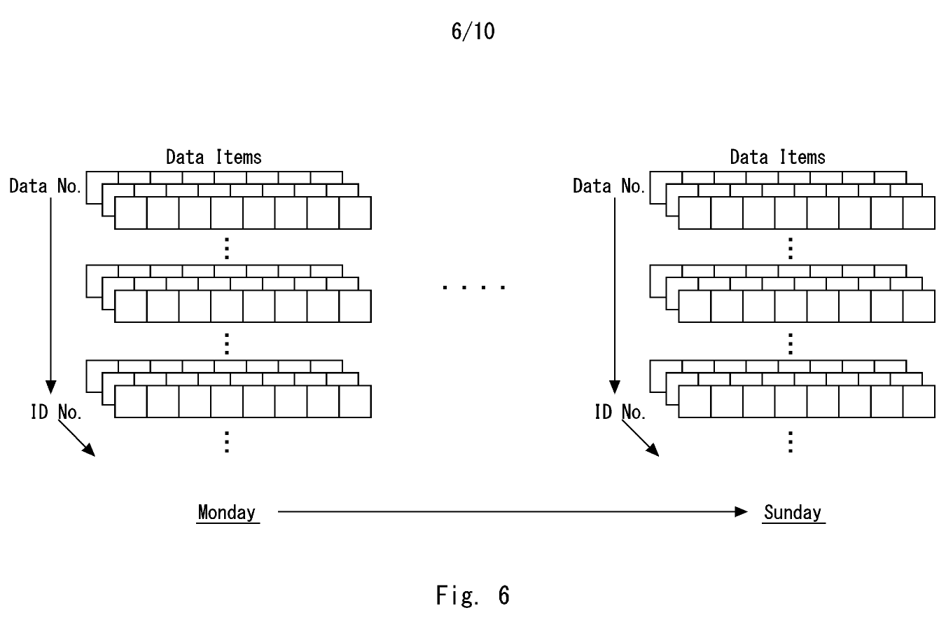

FIG. 6 illustrates an image of measurement data classified for each day of the week. FIG. 6 illustrates an image in which measurement data generated on Monday is extracted from the measurement data illustrated in FIG. 5, and in addition, FIG. 6 illustrates an image in which measurement data generated on Sunday is extracted. Although description regarding Tuesday to Saturday is omitted, measurement data is extracted similarly to those of Monday and Sunday.

Returning to FIG. 4, the second intermediate processing unit 21_2 generates statistical data related to the measurement data classified for each day of the week (S22).

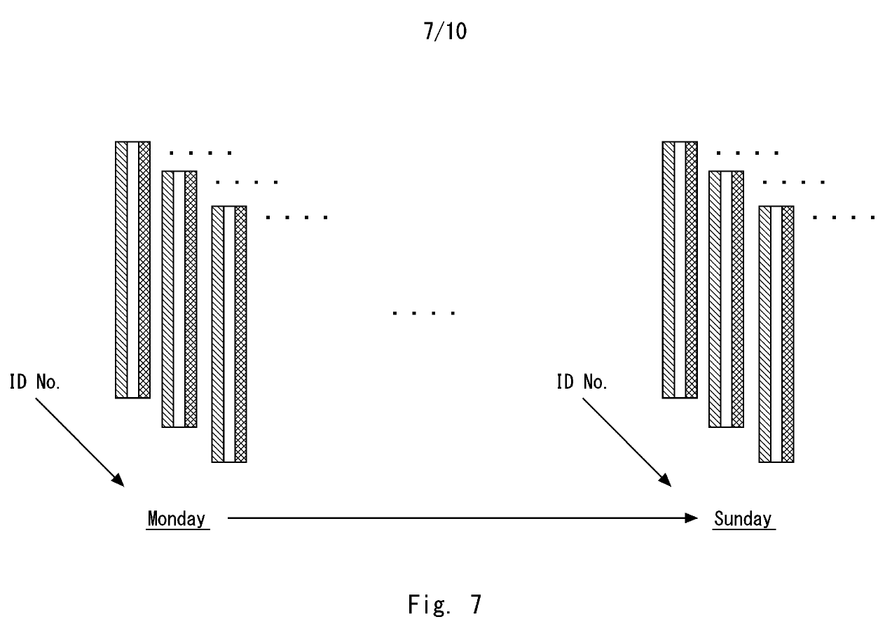

FIG. 7 illustrates an image regarding the statistical data. FIG. 7 illustrates that three types of statistical data are generated for each data item, on each day of the week. However, the number of types of statistical data is not limited to three, and three or more types may be generated, or one or two types may be generated. In FIG. 7, rectangles having the same pattern indicate a single piece of statistical data. The statistical data is generated for each subject. The statistical data may be, for example, an average, a maximum value, a minimum value, a deviation, a standard deviation, or the like of the measurement data. The statistical data may be a variation rate obtained by dividing the deviation by the average. The second intermediate processing unit 21_2 may generate a different type of statistical data for each data item or may generate the different number of pieces of statistical data for each data item.

Here, in a case of generating the statistical data, the second intermediate processing unit 21_2 may apply weighting to each piece of the measurement data. For example, in a case of calculating the average as the statistical data, the second intermediate processing unit 21_2 may multiply the measurement data by a larger value as a weight, as the data is newer. As the value of the weight increases, an influence of the measurement data after multiplication on the statistical data increases. That is, as the date is newer, the influence on the statistical data becomes larger. For example, a monotonically increasing value may be used as a weighting value, or a non-linearly increasing value may be used as a weighting value. Alternatively, a weighting value on only a specific day of the week may be larger or smaller than those on other days of the week.

Returning to FIG. 4, the generation unit 21 performs analysis related to the statistical data (S23). In other words, the generation unit 21 generates analysis data indicating an analysis result.

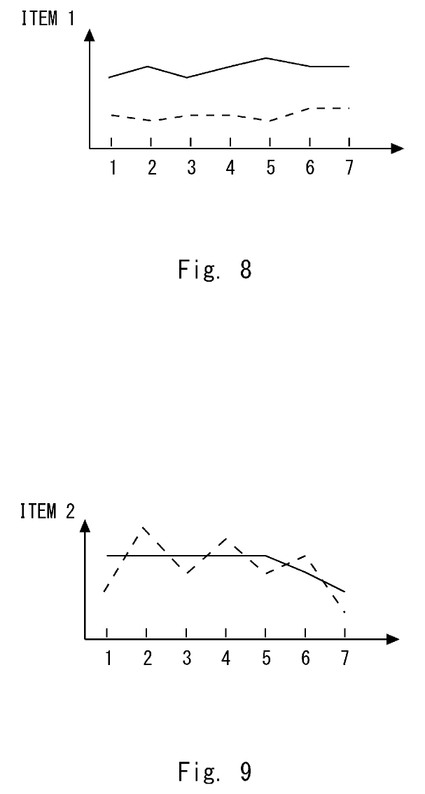

FIG. 8 illustrates an example of the analysis data generated by the generation unit 21. Numerical values of 1 to 7 on the horizontal axis indicate days of the week. For example, 1 may be Monday, 2 may be Tuesday, 3 may be Wednesday, 4 may be Thursday, 5 may be Friday, 6 may be Saturday, and 7 may be Sunday. The vertical axis indicates a magnitude of a numerical value of the data item. As the magnitude of the numerical value of the data item (item 1), for example, an average of the measurement data on each day of the week may be used.

The generation unit 21 may generate the analysis data using data regarding a disease risk known in advance, together with the statistical data. The data regarding the disease risk may be, for example, data related to whether the subject has frailty. The frailty does not correspond to a disease, but may be, for example, a state where muscle strength or the like is weakened. For example, in a case where an answer result of the subject to a questionnaire item determined in advance is a result indicating that the answer does not apply to any item, it may be determined that the subject does not have frailty, and in a case where the answer applies to one or more items, it may be determined that the subject has frailty.

In the analysis data in FIG. 8, data of the subject who does not have frailty is expressed by a solid line, and data of the subject who has frailty is expressed by a broken line. The example of the analysis data in FIG. 8 indicates a state in which there is a clear difference in a variation of the data item in one-week cycle between the subject who has frailty and the subject who does not have frailty. For example, the solid line and the broken line do not intersect, and in addition, a minimum value indicated by the solid line does not fall below a maximum value indicated by the broken line. Although the data of the subject who does not have frailty and the data of the subject who has frailty are illustrated as a single graph in FIG. 8, a single graph may be illustrated for each subject.

Next, FIG. 9 also illustrates an example of the analysis data generated by the generation unit 21. Numerical values of 1 to 7 on the horizontal axis indicate the days of the week as in FIG. 8. The vertical axis indicates a magnitude of a numerical value of a data item (item 2) different from that in FIG. 8. As the magnitude of the numerical value of the data item, as in FIG. 8, the average of the measurement data on each day of the week may be used.

In the analysis data in FIG. 9, the data of the subject who does not have frailty is expressed by a solid line, and the data of the subject who has frailty is expressed by a broken line. The example of the analysis data in FIG. 9 indicates a state in which there is no clear difference in the variation of the data item in one-week cycle between the subject who has frailty and the subject who does not have frailty. For example, the broken line repeatedly exceeds and falls below the solid line. Moreover, in a case where weekly averages of the solid line and the broken line are calculated, a result is assumed such that there is no large difference between the values. In the example in the analysis data in FIG. 9, regarding a difference between the subject who does not have frailty and the subject who has frailty, for example, the solid line indicating the subject who does not have frailty has a smaller value variation than the broken line. Although the data of the subject who does not have frailty and the data of the subject who has frailty are illustrated as a single graph in FIG. 9, a single graph may be illustrated for each subject.

The generation unit 21 generates analysis data having various shapes, in addition to the shapes illustrated in FIGS. 8 and 9. Next, the training unit 22 performs machine learning using the analysis data and the disease risk of the subject as the training data (S24). For example, the training unit 22 may use a neural network to perform machine learning. For example, the training unit 22 may train a value set for a weight of a synapse in the neural network, using the training data. The training unit 22 performs machine learning using the analysis data and the disease risk of the subject as the training data so as to generate a training model that estimates the disease risk of the subject, by inputting the analysis data of the subject.

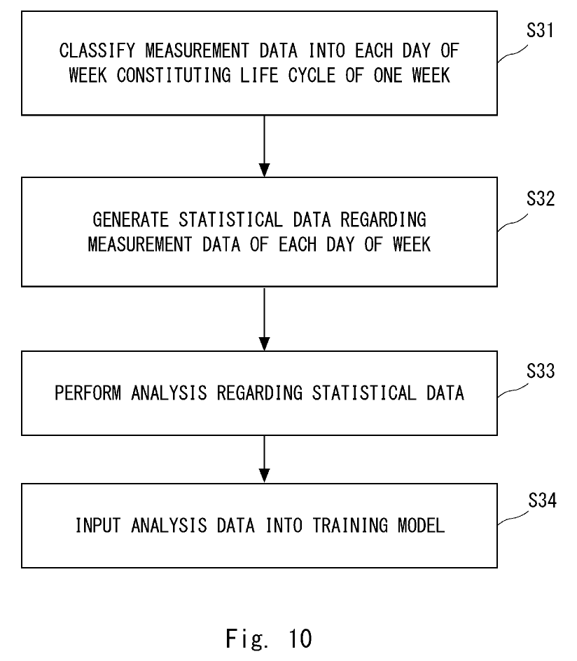

Next, a flow of disease risk estimation processing will be described with reference to FIG. 10. Since steps S31 to S33 are processes for generating the analysis data and similar to steps S21 to S23 in FIG. 4, detailed description thereof is omitted.

Next, the estimation unit 23 inputs the analysis data generated in step S33 into the training model generated in the process in FIG. 4 and receives information regarding the disease risk (S34).

As described above, the training device 20 uses the measurement data regarding the subject and the analysis data indicating the variation of the measurement data in the life cycle as the training data. As a result, for example, in a case where statistical data in a certain period is used without considering the variation of the life cycle, the disease risk can be estimated, even with the measurement data illustrated in FIG. 9 with no significant difference.

Although an example of using the analysis data as the training data has been described, the present disclosure is not limited to this. For example, as the training data, the statistical data used to generate the analysis data may be used, or the measurement data used to generate the statistical data may be used. That is, the training unit 22 may perform machine learning using the measurement data or the analysis data and the disease risk as the training data, to generate the statistical data or to generate the analysis data. As a result, the estimation unit 23 can receive the disease risk by inputting the measurement data or the statistical data.

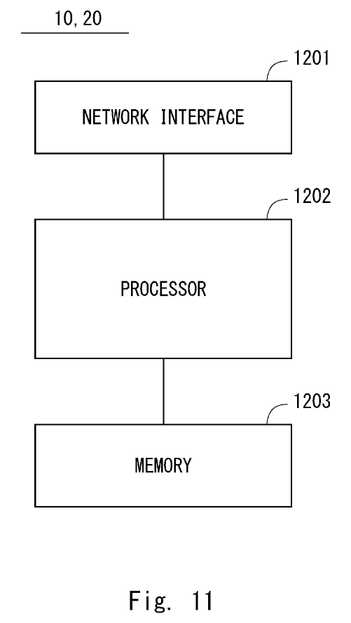

FIG. 11 is a block diagram illustrating a configuration example of the training device 10 and the training device 20 (hereinafter, referred to as training device 10 and the like). Referring to FIG. 11, the training device 10 and the like include a network interface 1201, a processor 1202, and a memory 1203. The network interface 1201 may be used to communicate with network nodes. The network interface 1201 may include, for example, a Network Interface Card (NIC) conforming to IEEE 802.3 series. IEEE represents Institute of Electrical and Electronics Engineers.

The processor 1202 performs the processing of the training device 10 and the like described using the flowcharts, by reading software (computer program) from the memory 1203 and executing the software. The processor 1202 may be, for example, a Micro Processor Unit (MPU) or a Central Processing Unit (CPU). The processor 1202 may include a plurality of processors.

The memory 1203 is constituted by a combination of a volatile memory and a nonvolatile memory. The memory 1203 may include a storage arranged away from the processor 1202. In this case, the processor 1202 may access the memory 1203 via an Input/Output (I/O) interface (not illustrated).

In the example in FIG. 11, the memory 1203 is used to store a software module group. The processor 1202 can perform the processing of the training device 10 and the like by reading the software module group from the memory 1203 and executing the software module group.

As described with reference to FIG. 11, each of the processors included in the training device 10 and the like executes one or a plurality of programs including an instruction group for causing a computer to perform an algorithm described with reference to the drawings.

In the example described above, the program includes a group of commands (or software codes) for causing a computer to execute the one or more functions described in the example embodiments in a case where the program is read by the computer. The program may be stored in a non-transitory computer-readable medium or a tangible storage medium. As an example and not by way of limitation, a computer-readable medium or tangible storage medium includes a Random-Access Memory (RAM), a Read-Only Memory (ROM), a flash memory, a Solid-State Drive (SSD), or other memory technologies, a CD-ROM, a Digital Versatile Disc (DVD), a Blu-ray (registered trademark) disk, or other optical disk storages, a magnetic cassette, a magnetic tape, a magnetic disk storage, or other magnetic storage devices. The program may be transmitted through a transitory computer-readable medium or a communication medium. As an example and not by way of limitation, the transitory computer-readable medium or the communication medium includes propagated signals in electrical, optical, acoustic, or any other form.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each example embodiment can be appropriately combined with other example embodiments.

Each drawing is merely illustrative for describing one or more example embodiments. Each of the drawings is not associated with only one specific example embodiment, but may be associated with one or more other example embodiments. As those of ordinary skill in the art will appreciate, various features or steps described with reference to any one of the drawings may be combined with features or steps illustrated in one or more other drawings, for example, to create an example embodiment that is not explicitly illustrated nor described. All of the features or steps illustrated in any one of the drawings for describing illustrative example embodiments are not necessarily mandatory, and some features or steps may be omitted. The order of the steps described in any one of the drawings may be changed as appropriate.

Some or all of the above example embodiments can also be described as the following Supplementary Notes, but are not limited to the following.

### Supplementary Note 1

A training device including:

a generation unit configured to generate analysis data indicating a variation of measurement data in a life cycle, based on a timing at which the measurement data regarding a first subject whose disease risk is known in advance is generated; and

a training unit configured to machine learn a training model for estimating the disease risk using the analysis data as an input, using the disease risk of the

first subject known in advance and the analysis data regarding the first subject as training data.

### Supplementary Note 2

The training device according to supplementary note 1, in which the life cycle is a cycle including seven days of a week.

### Supplementary Note 3

The training device according to supplementary note 2, in which the generation unit classifies each piece of the measurement data into any one of the days of the week, based on a day of the week at which the measurement data is generated.

### Supplementary Note 4

The training device according to supplementary note 3, in which the generation unit generates the analysis data indicating a variation of a value obtained by statistically processing the measurement data for each day of the week.

### Supplementary Note 5

The training device according to supplementary note 2, in which the generation unit includes a first intermediate processing unit that classifies the measurement data regarding the first subject into each day of the week and a second intermediate processing unit that statistically processes each piece of the measurement data classified into each day of the week.

### Supplementary Note 6

The training device according to any one of supplementary notes 1 to 5, in which the generation unit further includes a weight processing unit that increases a weight, as a timing at which the measurement data is generated is newer.

### Supplementary Note 7

The training device according to any one of supplementary notes 1 to 6, further including an estimation unit configured to input the measurement data regarding a second subject whose disease risk is estimated into the machine-learned training model and estimate the disease risk of the second subject.

### Supplementary Note 8

A training device including:

an acquisition unit configured to acquire a disease risk regarding a first subject known in advance and measurement data regarding the first subject; and

a training unit configured to train a variation of the measurement data in a life cycle based on a timing at which the measurement data is generated, using the disease risk regarding the first subject known in advance and the measurement data regarding the first subject as training data and machine learn a training model that estimates the disease risk.

### Supplementary Note 9

A training method including:

generating analysis data indicating a variation of measurement data in a life cycle, based on a timing at which the measurement data regarding a first subject whose disease risk is known in advance is generated; and

estimating the disease risk using the analysis data as an input, using the disease risk of the first subject known in advance and the analysis data regarding the first subject as training data.

### Supplementary Note 10

A training method including:

acquiring a disease risk regarding a first subject known in advance and measurement data regarding the first subject; and

machine learning a variation of the measurement data in a life cycle based on a timing at which the measurement data is generated, using the disease risk regarding the first subject known in advance and the measurement data regarding the first subject as training data and machine learning a training model that estimates the disease risk.

### Supplementary Note 11

A program for causing a computer to execute:

generating analysis data indicating a variation of measurement data in a life cycle, based on a timing at which the measurement data regarding a first subject whose disease risk is known in advance is generated; and

estimating the disease risk using the analysis data as an input, using the disease risk of the first subject known in advance and the analysis data regarding the first subject as training data.

### Supplementary Note 12

A program for causing a computer to execute:

acquiring a disease risk regarding a first subject known in advance and measurement data regarding the first subject; and

machine learning a variation of the measurement data in a life cycle based on a timing at which the measurement data is generated, using the disease risk regarding the first subject known in advance and the measurement data regarding the first subject as training data and estimating the disease risk.

Some or all of the elements (such as configurations and functions, for example) described in Supplementary Notes 2 to 7 dependent on Supplementary Note 1 may be dependent on Supplementary Notes 8 to 12 as well with dependent relationships similar to those of Supplementary Notes 2 to 7. Some or all of the elements described in any Supplementary Note may be applied to various types of hardware, software, recording means for recording software, systems, and methods.

1. A training device comprising:
at least one memory storing instructions; and
at least one processor configured to execute the instructions to:
generate analysis data indicating a variation of measurement data in a life cycle including days of a week, the measurement data being life log data regarding a first subject and being associated with a date and time including a day of the week, the generating including, for each day of the week, statistically processing the measurement data generated on the day of the week to calculate a statistical amount of the measurement data for the day of the week; and
machine learn a training model that estimates a disease risk of a subject from the analysis data, by using a disease risk regarding the first subject known in advance and the analysis data regarding the first subject as training data.

2. The training device according to claim 1, wherein the at least one processor is further configured to execute the instructions to:
classify each piece of the measurement data into one of the days of the week based on the date and time at which the measurement data is generated.

3. The training device according to claim 1, wherein
the statistically processing includes applying a larger weight to a piece of the measurement data generated at a newer timing than to a piece of the measurement data generated at an older timing.

4. The training device according to claim 1, wherein the at least one processor is further configured to execute the instructions to:
generate analysis data regarding a second subject whose disease risk is to be estimated from measurement data regarding the second subject in the life cycle;
input the analysis data regarding the second subject into the machine-learned training model; and
estimate a disease risk of the second subject.

5. The training device according to claim 1, wherein
the measurement data includes at least one of a number of steps, a moving speed, a walking time, a stride length, a pitch, a walking distance, Metabolic Equivalent of Tasks (METs), total calorie consumption, a lying time, and a sitting time of the first subject.

6. The training device according to claim 1, wherein
the disease risk is a risk of at least one of diabetes, hypertension, and frailty.

7. A training method comprising:
generating, by a computer, analysis data indicating a variation of measurement data in a life cycle including days of a week, the measurement data being life log data regarding a first subject and being associated with a date and time including a day of the week, the generating including, for each day of the week, statistically processing the measurement data generated on the day of the week to calculate a statistical amount of the measurement data for the day of the week; and
machine learning, by the computer, a training model that estimates a disease risk of a subject from the analysis data, by using a disease risk regarding the first subject known in advance and the analysis data regarding the first subject as training data.

8. The training method according to claim 7, further comprising:
classifying, by the computer, each piece of the measurement data into one of the days of the week based on the date and time at which the measurement data is generated.

9. The training method according to claim 7, wherein
the statistical processing includes applying a larger weight to a piece of the measurement data generated at a newer timing than to a piece of the measurement data generated at an older timing.

10. The training method according to claim 7, further comprising:
generating, by the computer, analysis data regarding a second subject whose disease risk is to be estimated from measurement data regarding the second subject in the life cycle;
inputting, by the computer, the analysis data regarding the second subject into the machine-learned training model; and
estimating, by the computer, a disease risk of the second subject.

11. The training method according to claim 7, wherein
the measurement data includes at least one of a number of steps, a moving speed, a walking time, a stride length, a pitch, a walking distance, Metabolic Equivalent of Tasks (METs), total calorie consumption, a lying time, and a sitting time of the first subject.

12. The training method according to claim 7, wherein
the disease risk is a risk of at least one of diabetes, hypertension, and frailty.

13. A non-transitory computer-readable medium storing a program for causing a computer to execute:
generating analysis data indicating a variation of measurement data in a life cycle including days of a week, the measurement data being life log data regarding a first subject and being associated with a date and time including a day of the week, the generating including, for each day of the week, statistically processing the measurement data generated on the day of the week to calculate a statistical amount of the measurement data for the day of the week; and
machine learning a training model that estimates a disease risk of a subject from the analysis data, by using a disease risk regarding the first subject known in advance and the analysis data regarding the first subject as training data.

14. The non-transitory computer-readable medium according to claim 13, wherein
the program further causes the computer to classify each piece of the measurement data into one of the days of the week based on the date and time at which the measurement data is generated.

15. The non-transitory computer-readable medium according to claim 13, wherein
the program further causes the computer, in the statistical processing, to apply a larger weight to a piece of the measurement data generated at a newer timing than to a piece of the measurement data generated at an older timing.

16. The non-transitory computer-readable medium according to claim 13, wherein the program further causes the computer to:
generate analysis data regarding a second subject whose disease risk is to be estimated from measurement data regarding the second subject in the life cycle;
input the analysis data regarding the second subject into the machine-learned training model; and
estimate a disease risk of the second subject.

17. The non-transitory computer-readable medium according to claim 13, wherein
the program causes the computer to use the measurement data that includes at least one of a number of steps, a moving speed, a walking time, a stride length, a pitch, a walking distance, METs, total calorie consumption, a lying time, and a sitting time of the first subject.

18. The non-transitory computer-readable medium according to claim 13, wherein
the disease risk is a risk of at least one of diabetes, hypertension, and frailty.
